(19) United States
(12) Patent Application Publication
(10) Pub. No.: US 2026/0355020 A1
(43) Pub. Date: Nov. 12, 2026

(54) Nasal Discharge Stopper

(71) Applicant: Keith Goldstein, Evanston, IL (US)

(72) Inventor: Keith Goldstein, Evanston, IL (US)

(21) Appl. No.: 19/201,256

(22) Filed: May 7, 2025

(51) Int. Cl. A61F 13/20

(52) U.S. Cl. CPC A61F 13/2005

(57) ABSTRACT

A double-ended, curved, flexible plastic handle is fitted with an absorbent cotton swab at each end. The device is sized so the cotton swabs will fit snugly into human nostrils, while the flexible plastic handle will hold cotton swabs in place. The device will collect nasal discharge without requiring the use of hands to reduce discomfort, inconvenience, and transmission of pathogens.

### FIELD

The present invention relates to a device to improve sanitary treatment of nasal discharge. This disposable device allows for convenient and sanitary treatment of nasal discharge to reduce discomfort, inconvenience, and transmission of pathogens in said nasal discharge.

### BACKGROUND

Nasal discharge, such as mucus, blood, and other fluids, causes discomfort, inconvenience, and transmission of pathogens when people use their hands or other body parts to wipe the discharge. Facial tissues that are commonly used to catch discharge are permeable and result in nasal discharge seeping onto people's hands. Nasal discharge often drips onto surfaces if a person cannot catch it quickly enough. Nasal discharge also drips down a person's face causing discomfort and inconvenience.

### SUMMARY

By providing a device that will fit snugly into human nostrils, with absorbent cotton swabs on each end, the discomfort, inconvenience, and transmission of pathogens due to nasal discharge will be reduced. This is an improvement over prior methods of handling nasal discharge. The device may be inserted into the nostrils and then a person can use his hands freely, without the need to frequently wipe or contain the discharge.

### BRIEF DESCRIPTION OF THE DRAWING

The novel features and advantages of the invention will be better understood upon a reading of the following detailed description, in conjunction with the accompanying drawing, in which:

FIG. 1 is an elevational view of a device according to the invention, which stops nasal discharge.

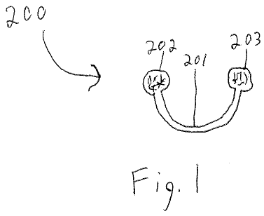

### DETAILED DESCRIPTION

In the following detailed description, the device according to the teachings for this application in the form of a nasal discharge stopper will be described by the embodiments.

Referring now to the drawing, there is illustrated a device designated generally by reference number 200. The device includes as its principal components a curved stick-like handle 201 preferably constructed of a flexible, break-resistant plastic and having two opposed ends to which cotton swabs 202 and 203 are attached such as by a suitable adhesive. It should be noted that the curvature of the plastic handle will be flexible enough to adapt to variations in individual human nostril widths and sizes.

The device 200 will offer considerable convenience in the treatment of nasal discharge, and offers considerable advantages over the alternatives, such as facial tissue.

While the present invention has been described in connection with preferred embodiments, it will be apparent to those skilled in the art that many changes and modifications may be made without departing from the true spirit and scope of the invention. Accordingly, it is intended by the appended claims to cover all such changes and modifications which come within the spirit and scope of the invention.

The invention claimed is:

1. A device for treatment and stoppage of nasal discharge, comprising of a thin, curved, flexible handle having two opposed ends with swabs affixed to each end.

2. The device of claim 1 wherein each end of said handle is provided with a swab.

3. The device of claim 1 wherein said swab is formed of cotton.

4. The device of claim 1 wherein said handle is formed from a flexible, break-resistant plastic.

5. The device of claim 1 wherein said handle is approximately 4.25 cm in length.

6. The device of claim 1 wherein the curve in said handle is at the midpoint of said handle.
